SEQUENCE LISTING (1 sequence)

SEQ ID NO: 1 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2025/0075276 A1
(43) Pub. Date: Mar. 6, 2025

(54) METHODS FOR SELECTING SUBJECTS AND TREATING CANCER WITH IL-2 THERAPY

(71) Applicant: Synthorx, Inc., La Jolla, CA (US)

(72) Inventors: Kirk BEEBE, Durham, NC (US); Joel EISNER, Durham, NC (US); Gregory MAYHEW, Durham, NC (US); Marcos MILLA, Carlsbad, CA (US); Jill MOONEY, Carlsbad, CA (US)

(73) Assignee: Synthorx, Inc., La Jolla, CA (US)

(21) Appl. No.: 18/727,910

(22) PCT Filed: Jan. 12, 2023
(86) PCT No.: PCT/US2023/060591

§ 371 Date: Jul. 10, 2024

Related U.S. Application Data
(60) Provisional application No. 63/299,113, filed on Jan. 13, 2022.

(51) Int. Cl. C12Q 1/6886; A61K 38/20; A61P 35/00; G16B 25/10

(52) U.S. Cl. CPC C12Q 1/6886; A61K 38/2013; A61P 35/00; G16B 25/10; C12Q 2600/106; C12Q 2600/158

(57) ABSTRACT

Described herein are methods for treating a cancer in a subject, the method comprising administering an IL-2 therapy to the subject, wherein in a sample from the subject, a set of markers has elevated expression or decreased expression.

### CROSS-REFERENCE TO RELATED APPLICATIONS

This application claims the benefit of priority of U.S. Provisional Patent Application No. 63/299,113, filed Jan. 13, 2022, which is incorporated by reference herein for all purposes.

### INTRODUCTION AND SUMMARY

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+ T and B lymphocytes, monocytes, macrophages, endothelial cells, and other tissue residents.

Interleukin 2 (IL-2) stimulates proliferation and survival of T and NK cells. In some instances, IL-2 signaling is used to modulate T cell responses and subsequently for treatment of a cancer. After its discovery, immunooncologists recognized the potential for IL-2 to generate “lymphokine-activated killer cells (LAKs),” later identified as CD8+ T effector and NK cells for cancer therapy. Subsequent to the discovery and use of IL-2 in immunooncology, a dramatic increase has occurred in the understanding of the tumor and immune microenvironment. These insights have led to the development of a plethora of therapeutic approaches for clinical use, such as modulators of T cell response including checkpoint inhibitory antibodies, T cell receptor engager/stimulatory antibodies, suppressors of regulatory T cells and myeloid-derived suppressor cells (MDSCs), as well as adoptive cell therapies, oncolytic viruses, vaccines, TLR agonists and more. While modulation of PD-1 and CTLA-4 with immune checkpoint inhibitors (ICI) has shown responses in a significant fraction of patients, a need remains to better understand the mechanisms driving resistance to such therapies.

IL-2 has emerged as an agent with potential for overcoming such resistance, with multiple IL-2 drugs moving through clinical trials as single agents or in combination with checkpoint inhibitors and other targeted immune-oncology agents. Examples include NKTR-214, a form of IL-2 reversibly conjugated with multiple PEG chains; PD1-IL2v, a programmed cell death protein 1 (PD-1) mAb/IL-2v fusion variant; R06874281, a fusion IL-2 variant; cergutuzumab amunaleukin, a fusion IL-2 variant; R06874281, a fibroblast activation protein (FAP) mAb/IL-2v fusion variant; cergutuzumab amunaleukin, a carcinoembryonic antigen (CEA) mAb/IL-2v fusion variant; and ALKS-4230, a fusion of IL-2 to the extracellular domain of the alpha chain of its receptor.

The present disclosure describes pre-treatment tumor molecular analysis, including RNA sequencing (RNAseq), combined with clinical annotation from a single-institution retrospective cohort of metastatic RCC patients who underwent treatment as part of an IL-2 clinical program. Genomic characteristics common to both IL-2 and anti-PD-(L)1 therapy response, as well as unique to each are provided.

In one aspect, provided herein are methods for treating a cancer in a subject, the method comprising administering an IL-2 therapy to the subject, wherein a sample from the subject shows certain characteristics with respect to immune markers, myeloid inflammation markers, or other classification markers.

Described herein are methods for treating a cancer in a subject, the method comprising administering an IL-2 therapy to the subject, wherein in a sample from the subject, a set of markers has elevated expression or decreased expression. Also described herein are methods for treating a cancer in a subject, the method comprising selecting the subject at least in part on the basis that in a sample from the subject, a set of markers has elevated expression or decreased expression; and administering an IL-2 therapy to the subject. Further described herein are methods for selecting a subject with a cancer for an IL-2 therapy, the method comprising selecting the subject at least in part on the basis that in a sample from the subject, a set of markers has elevated expression or decreased expression.

Exemplary embodiments include the following.

Embodiment 1 is a method for treating a cancer in a subject, the method comprising: administering an IL-2 therapy to the subject, wherein in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1.

Embodiment 2 is a method for treating a cancer in a subject, the method comprising: selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1; and administering an IL-2 therapy to the subject.

Embodiment 3 is a method for selecting a subject with a cancer for an IL-2 therapy, the method comprising: selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1.

Embodiment 4 is the method for the immediately preceding embodiment, further comprising: obtaining the sample from the subject.

Embodiment 5 is the method of any one of embodiments 3-4, further comprising: extracting RNA from the sample.

Embodiment 6 is the method of any one of embodiments 3-5, further comprising: measuring expression of the set of upregulated classification markers and/or the set of downregulated classification markers in the sample.

Embodiment 7 is the method of the immediately preceding embodiment, wherein the measuring of expression comprises sequencing RNA from the sample, conducting RT-qPCR with RNA from the sample, conducting a microarray analysis of the sample, or conducting a proteomic analysis of the sample.

Embodiment 8 is the method of any one of the preceding embodiments, wherein the set of upregulated classification markers has elevated expression.

Embodiment 9 is the method of the immediately preceding embodiment, wherein the set of upregulated classification markers comprises one of, two of, at least two of, three of, at least three of, four of, at least four of, five of, at least five of, six of, at least six of, seven of, at least seven of, eight of, at least eight of, nine of, at least nine of, ten of, at least ten of, 11 of, at least 11 of, 12 of, at least 12 of, 13 of, at least 13 of, 14 of, at least 14 of, 15 of, at least 15 of, 16 of, at least 16 of, 17 of, at least 17 of, 18 of, at least 18 of, 19 of, at least 19 of, 20 of, at least 20 of, 21 of, at least 21 of, 22 of, at least 22 of, 23 of, at least 23 of, 24 of, at least 24 of, 25 of, at least 25 of, 26 of, at least 26 of, 27 of, at least 27 of, 28 of, at least 28 of, 29 of, at least 29 of, 30 of, at least 30 of, or 31 of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644.

Embodiment 10 is the method of any one of the preceding embodiments, wherein the set of downregulated classification markers has decreased expression.

Embodiment 11 is the method of the immediately preceding embodiment, wherein the set of downregulated classification markers comprises one of, two of, at least two of, three of, at least three of, four of, at least four of, five of, at least five of, six of, at least six of, seven of, at least seven of, eight of, at least eight of, or nine of: HLF, COL4A4, SLITRK4, SHISA9, IGSF9, TJP3, ZNF853, B4GALNT3, and ATP6V1B1.

Embodiment 12 is the method of any one of the preceding embodiments, wherein the set of upregulated classification markers comprises at least one of: GZMA, CD300E, LILRA5, IL1RN, CCL2, and CXCL2.

Embodiment 13 is the method of the immediately preceding embodiment, wherein the set of upregulated classification markers comprises two of, at least two of, three of, at least three of, four of, at least four of, five of, at least five of, or six of: GZMA, CD300E, LILRA5, IL1RN, CCL2, and CXCL2.

Embodiment 14 is the method of any one of the preceding embodiments, wherein the set of upregulated classification markers comprises at least one of: FPR2, S100A9, IL1RN, and CCL2.

Embodiment 15 is the method of the immediately preceding embodiment, wherein the set of upregulated classification markers comprises two of, at least two of, three of, at least three of, or four of: FPR2, S100A9, IL1RN, and CCL2.

Embodiment 16 is the method of any one of the preceding embodiments, wherein the set of upregulated classification markers comprises at least X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

Embodiment 17 is the method of any one of the preceding embodiments, wherein the set of upregulated classification markers comprises at least 2X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

Embodiment 18 is the method of any one of the preceding embodiments, wherein the set of upregulated classification markers comprises at least 3X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

Embodiment 19 is the method of any one of the preceding embodiments, wherein the cancer is renal cancer.

Embodiment 20 is the method of any one of the preceding embodiments, wherein the cancer is renal cell carcinoma.

Embodiment 21 is the method of any one of the preceding embodiments, wherein the cancer is renal cell carcinoma of subtype clear cell B (ccB).

Embodiment 22 is the method of any one of the preceding embodiments, wherein the cancer is metastatic.

Embodiment 23 is the method of any one of the preceding embodiments, wherein the subject has not received any prior IL-2 therapy.

Embodiment 24 is the method of any one of the preceding embodiments, wherein the IL-2 therapy comprises a polypeptide having at least 80%, 85%, 90%, 95%, 96%, 97%, 98%, 99%, or 100% sequence identity to SEQ ID NO: 1.

Embodiment 25 is the method of any one of the preceding embodiments, wherein the IL-2 therapy comprises aldesleukin.

Embodiment 26 is the method of any one of the preceding embodiments, the IL-2 therapy is administered to the subject by subcutaneous administration.

Embodiment 27 is the method of any one of the preceding embodiments, the IL-2 therapy is administered to the subject by intravenous administration.

Embodiment 28 is the method of any one of the preceding embodiments, further comprising refraining from administering a PD-1-targeting therapy or PD-L1-targeting therapy to the subject.

Embodiment 29 is the method of any one of the preceding embodiments, further comprising refraining from administering a PD-1-targeting therapy to the subject.

Embodiment 30 is the method of any one of the preceding embodiments, wherein the sample comprises a tumor tissue.

Embodiment 31 is the method of any one of the preceding embodiments, wherein the sample comprises a primary tumor tissue or a metastatic tumor tissue.

Embodiment 32 is the method of any one of the preceding embodiments, wherein the sample comprises a formalin-fixed paraffin embedded (FFPE) tumor tissue.

Embodiment 33 is the method of any one of the preceding embodiments, wherein the sample comprises immune tissue.

Embodiment 34 is the method of any one of the preceding embodiments, wherein the set of upregulated classification markers has elevated expression and/or the set of downregulated classification markers has decreased expression according to nearest centroid analysis

Embodiment 35 is the method of the immediately preceding embodiment, wherein the nearest centroid analysis comprises determining first and second distances; the first distance is between a sample vector comprising expression values of the set of upregulated classification markers and/or the set of downregulated classification markers and a first reference centroid of the set of upregulated classification markers and/or the set of downregulated classification markers determined from expression values from IL-2 non-refractory subjects; and the second distance is between the sample vector and a second reference centroid determined from expression values of the set of upregulated classification markers and/or the set of downregulated classification markers from IL-2 refractory subjects

Embodiment 36 is the method of any one of the preceding embodiments, wherein elevated expression and/or decreased expression are determined using logarithmic (e.g., log 2) median-centered expression values for the set of upregulated classification markers and/or the set of downregulated classification markers.

Embodiment 37 is an IL-2 therapy for use in the method of any one of the preceding embodiments.

Embodiment 38 is use of an IL-2 therapy for the manufacture of a medicament for the method of any one of embodiments 1-36.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a heatmap showing expression of sets of cell-associated genes and individual genes (rows) in IL-2 pretreatment tumors (columns). FIG. 1B shows boxplots of sets of cell-associated genes and individual genes in pretreatment tumor samples with open circles colored by clinical response.

FIG. 2A shows a heatmap showing expression of 40 markers (rows) in the classifier in IL-2 pretreatment tumors (columns). FIG. 2B shows a boxplots of selected markers in pretreatment tumor samples with open circles colored by clinical response. FIG. 2C shows a volcano plot of overrepresentation analysis of the 40 markers in the classifier and the associated gene sets.

FIG. 3A shows a Kaplan-Meier plot of TCGA KIRC tumor samples (n=253) based on the 40-marker IL-2 response classifier of Example 2. FIG. 3B shows a Kaplan-Meier plot of TCGA KIRC tumor samples (n=253) based on a canonical intrinsic subtype of RCC (the immune-high ccB). FIG. 3C shows a Kaplan-Meier plot of tumor samples from RCC patients treated with HD-IL-2 (n=36) based on a canonical intrinsic subtype of RCC (the immune-high ccB) along with a clinical response comparison.

FIG. 4A shows heatmaps showing expression of sets of cell-associated genes and individual genes (rows) in IL-2 pretreatment tumors (columns in left heat map) and anti-PD-1 (right heat map). FIG. 4B shows statistical results for the sets of cell-associated genes and individual genes, comparing pretreatment responsive tumors (CR+PR) to non-responsive tumors (SD+PD) for IL-2 and anti-PD-1-treated patients. FIG. 4C shows heatmaps showing expression of markers (rows) in IL-2 pretreatment (columns, left heat map) and anti-PD-1 (right) tumors. FIG. 4D shows statistical results for the markers comparing pretreatment responsive tumors (CR+PR) to non-responsive tumors (SD+PD) for IL-2 and anti-PD-1-treated patients.

FIG. 4E shows a scatter plot showing the relationship between the myeloid inflammation signature (Myeloid) and the T cell effector (Teff) signature for IL-2 and anti-PD-1 pre-treatment tumors, respectively.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “expression” refers to an RNA level or a protein level of a set of markers (i.e., at least one marker). In some embodiments, a marker comprises a single RNA or protein; in such instance, the expression is the RNA level or the protein level of the single RNA or protein. In some embodiments, a marker comprises a set of RNAs or proteins; in such instances, the expression is the average or median of levels of the RNAs or proteins.

As used herein, “decreased expression” refers to (i) expression of a set of markers (i.e., at least one marker) in a sample from a subject having a median or mean expression value lower than (e.g., statistically significantly lower than) expression of the same set of markers in a sample from another subject with a cancer that is refractory to an IL-2 therapy or a statistical threshold (e.g., mean or median) calculated from a population of samples from subjects with cancers refractory to an IL-2 therapy, or (ii) for a set of markers comprising a plurality of markers, expression of the set of markers in a sample from a subject being closer to a first reference centroid than a second reference centroid, wherein the first reference centroid is determined for the set of markers from an individual or population having a cancer that is not refractory to an IL-2 therapy and the second reference centroid is determined for the set of markers from an individual or population having a cancer that is refractory to the IL-2 therapy. In some embodiments, the expression values are logarithmic (e.g., log 2) median-centered expression values. Nearest centroid analysis is discussed in detail in Dabney, Bioinformatics 21:4148-4154 (2005), doi:10.1093/bioinformatics/bti681. Briefly, a vector (which can also be considered a set of coordinates in n dimensions where n is the number of markers in the set) is formed from each set of gene expression values to be compared, where the vectors formed from the set of markers from an individual or population having a cancer that is not refractory to an IL-2 therapy and the set of markers from an individual or population having a cancer that is refractory to the IL-2 therapy are the first and second reference centroids, respectively. Where populations are used, the centroids can be vectors of means or medians. The distance in n-dimensional space from the first and second reference centroids is determined for the vector formed from the expression of the set of markers in the sample from the subject (sample vector). If the sample vector is closer to the first reference centroid than to the second reference centroid, it indicates decreased expression. The nearest centroid approach can also be applied to determine decreased expression relative to a first set of markers and increased expression relative to a second set of markers simultaneously, using vectors/centroids containing both the first and second sets of markers. In such a case, if the sample vector is closer to the first reference centroid than to the second reference centroid, then it is considered to indicate both that there is decreased expression of the first set of markers and increased expression of the second set of markers. For each of the methods described and claimed herein, one who performs the method need not measure the expression of the set of markers in the other sample(s) from the other subject or population thereof; instead, the expression may be obtained by any means, such as being obtained from a database.

As used herein, “elevated expression” refers to (i) expression of a set of markers (i.e., at least one marker) in a sample from a subject having a median or mean expression value higher than (e.g., statistically significantly lower than) expression of the same set of markers in a sample from another subject with a cancer that is refractory to an IL-2 therapy or a statistical threshold (e.g., mean or median) calculated from a population of samples from subjects with cancers refractory to an IL-2 therapy, or (ii) for a set of markers comprising a plurality of markers, expression of the set of markers in a sample from a subject being closer to a first reference centroid than a second reference centroid, wherein the first reference centroid is determined for the set of markers from an individual or population having a cancer that is not refractory to an IL-2 therapy and the second reference centroid is determined for the set of markers from an individual or population having a cancer that is refractory to the IL-2 therapy. In some embodiments, the expression values are logarithmic (e.g., log 2) median-centered expression values. Nearest centroid analysis is as discussed above. The nearest centroid approach can also be applied to determine decreased expression relative to a first set of markers and increased expression relative to a second set of markers simultaneously, using vectors/centroids containing both the first and second sets of markers. In such a case, if the sample vector is closer to the first reference centroid than to the second reference centroid, then it is considered to indicate both that there is decreased expression of the first set of markers and increased expression of the second set of markers. For each of the methods described and claimed herein, one who performs the method need not measure the expression of the set of markers, in the other sample from the other subject; instead, the expression may be obtained by any means, such as being obtained from a database. For each of the methods described and claimed herein, one who performs the method need not measure the expression of the set of markers in the other sample(s) from the other subject or population thereof; instead, the expression may be obtained by any means, such as being obtained from a database.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2

Interleukin 2 (IL-2) is a pleiotropic type-1 cytokine whose structure comprises a 15.5 kDa four a-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2RT (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rτ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺CD25⁺ T cells suppressed the development of autoimmunity (Sakaguchi, et al., “Immunologic self-tolerance maintained by activated T cells expressing IL-2 receptor alpha-chains (CD25),” J. Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

### IL-2 Therapy

In some embodiments, the IL-2 therapy comprises a polypeptide having at least 80%, 85%, 90%, 95%, 96%, 97%, 98%, 99%, or 100% sequence identity to PTSSSTKKTQLQLEHLLLDLQMILNGINNYKNPKLTRMLTFKFYMPKKATELKHLQCLE EELKPLEEVLNLAQSKNFHLRPRDLISNINVIVLELKGSETTFMCEYADETATIVEFLNR WITFSQSIISTLT (SEQ ID NO: 1). In some embodiments, the IL-2 therapy that may be used in the methods provided herein comprises aldesleukin.

### Methods for Treatment and Methods for Selecting Subject for Treatment

In some embodiments, provided herein is a method for treating a cancer in a subject, the method comprising: administering an IL-2 therapy to the subject, wherein in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1.

In some embodiments, provided herein is a method for treating a cancer in a subject, the method comprising: selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1; and administering an IL-2 therapy to the subject.

In some embodiments, provided herein is a method for selecting a subject with a cancer for an IL-2 therapy, the method comprising: selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644; and the set of downregulated classification markers comprises at least one of HLF, COL4A4, SLITRK4, SHISA9, IGSF9, TJP3, ZNF853, B4GALNT3, and ATP6V1B1.

In some embodiments, any of the methods provided herein further comprises obtaining the sample from the subject. In some embodiments, the sample comprises a tumor tissue. In some embodiments, the sample comprises a primary tumor tissue or a metastatic tumor tissue. In some embodiments, the sample comprises a formalin-fixed paraffin embedded (FFPE) tumor tissue. In some embodiments, the sample comprises immune tissue.

In some embodiments, any of the methods provided herein further comprises extracting RNA from the sample. In some embodiments, any of the methods provided herein further comprises extracting DNA from the sample. In some embodiments, the extracting RNA from the sample and/or the extracting DNA from the sample comprises dual DNA/RNA extraction.

In some embodiments, any of the methods provided herein further comprises measuring expression of upregulated classification markers and/or downregulated classification markers. In some embodiments, the measuring of expression comprises performing an RNA expression analysis.

In some embodiments, the set of classification markers comprises one of, at least one of, two of, at least two of, three of, at least three of, four of, at least four of, five of, at least five of, six of, at least six of, seven of, at least seven of, eight of, at least eight of, nine of, at least nine of, ten of, at least ten of, 11 of, at least 11 of, 12 of, at least 12 of, 13 of, at least 13 of, 14 of, at least 14 of, 15 of, at least 15 of, 16 of, at least 16 of, 17 of, at least 17 of, 18 of, at least 18 of, 19 of, at least 19 of, 20 of, at least 20 of, 21 of, at least 21 of, 22 of, at least 22 of, 23 of, at least 23 of, 24 of, at least 24 of, 25 of, at least 25 of, 26 of, at least 26 of, 27 of, at least 27 of, 28 of, at least 28 of, 29 of, at least 29 of, 30 of, at least 30 of, or 31 of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644.

In some embodiments, the set of upregulated classification markers comprises one of, at least one of, two of, at least two of, three of, at least three of, four of, at least four of, five of, at least five of, or six of: GZMA, CD300E, LILRA5, IL1RN, CCL2, and CXCL2.

In some embodiments, the set of upregulated classification markers comprises one of, at least one of, two of, at least two of, three of, at least three of, or four of: FPR2, S100A9, IL1RN, and CCL2.

In some embodiments, the set of downregulated classification markers comprises one of, two of, at least two of, three of, at least three of, four of, at least four of, five of, at least five of, six of, at least six of, seven of, at least seven of, eight of, at least eight of, or nine of: HLF, COL4A4, SLITRK4, SHISA9, IGSF9, TJP3, ZNF853, B4GALNT3, and ATP6V1B1.

In some embodiments, decreased expression comprises expression of a set of markers (i.e., at least one marker), in a sample from a subject, being at least 10% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 20% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 30% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 40% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 50% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 60% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 70% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 80% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 90% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 95% lower than expression of the same set of markers, in another sample from another subject with a cancer that is refractory to an IL-2 therapy. In some embodiments, decreased expression comprises expression of a set of markers (i.e., at least one marker), in a sample from a subject, being at least 99% lower than expression of the same set of markers, in another sample from another subject with a cancer that is refractory to an IL-2 therapy. For each of the methods provided herein, one who performs the method need not measure the expression of the set of markers, in the other sample from the other subject; instead, the expression may be obtained by any means, such as being obtained from a database.

In some embodiments, elevated expression comprises expression of a set of markers (i.e., at least one marker), in a sample from a subject, being at least 10% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 20% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 30% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 40% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 50% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 60% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 70% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 80% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 90% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 95% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 100% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 200% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 300% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 400% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 500% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 600% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 700% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 800% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 900% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 1,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 2,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 3,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 4,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 5,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 6,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 7,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 8,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 9,000% higher than expression of the same set of markers, in another sample from another subject with a cancer that is refractory to an IL-2 therapy. In some embodiments, elevated expression comprises expression of a set of markers (i.e., at least one marker), in a sample from a subject, being at least 10,000% higher than expression of the same set of markers, in another sample from another subject with a cancer that is refractory to an IL-2 therapy. For each of the methods provided herein, one who performs the method need not measure the expression of the set of markers, in the other sample from the other subject; instead, the expression may be obtained by any means, such as being obtained from a database.

In some embodiments, the subject has not received any prior IL-2 therapy. In some embodiments, the subject has received prior IL-2 therapy.

In some embodiments, the method further comprises refraining from administering a PD-1-targeting therapy (such as an anti-PD-1 antibody, e.g., pembrolizumab, nivolumab, cemiplimab, atezolizumab, dostarlimab, durvalumab, or avelumab) or PD-L1-targeting therapy (such as an anti-PD-L1 antibody, e.g., atezolizumab, avelumab, or durvalumab) to the subject. In some embodiments, the method further comprises refraining from administering a PD-1-targeting therapy (such as an anti-PD-1 antibody, e.g., pembrolizumab, nivolumab, cemiplimab, atezolizumab, dostarlimab, durvalumab, or avelumab) to the subject. In some embodiments, the method further comprises refraining from administering a PD-L1-targeting therapy (such as an anti-PD-L1 antibody, e.g., atezolizumab, avelumab, or durvalumab) to the subject.

In some embodiments, provided herein is use of an IL-2 therapy as described herein for any of the methods for treating a cancer in a subject or methods for selecting a subject with a cancer disclosed herein. In some embodiments, provided herein is use of an IL-2 therapy as described herein for the manufacture of a medicament for any of the methods for treating a cancer in a subject or methods for secting a subject with a cancer disclosed herein.

The embodiments described in the following sections apply to any of the foregoing aspects.

### Cancer Types

In some embodiments, the cancer is renal cancer. In some embodiments, the cancer is renal cell carcinoma. In some embodiments, the cancer is clear cell renal cell carcinoma. In some embodiments, the cancer is non-clear cell renal cell carcinoma. In some embodiments, the non-clear cell renal cell carcinoma is chromophobe renal cell carcinoma, collecting duct renal cell carcinoma, multilocular cystic renal cell carcinoma, medullary carcinoma, mucinous tubular and spindle cell carcinoma, or neuroblastoma-associated renal cell carcinoma. In some embodiments, the cancer is transitional cell carcinoma, Wilms tumor, or renal sarcoma. In some embodiments, the cancer is renal cell carcinoma of subtype clear cell B (ccB). For a discussion of ccB renal cell carcinoma, see, e.g., Brannon et al., Genes Cancer 1(2):152-163 (2010) (doi: 10.1177/1947601909359929; available at <https://www.ncbi.nlm.nih.gov/pmc/articles/PMC2943630/>).

In some embodiments, the cancer is metastatic.

### Administration

### 1. Route

In some embodiments, the IL-2 therapy is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 therapy is administered to the subject by intravenous, subcutaneous, or intramuscular administration. In some embodiments, the IL-2 therapy is administered to the subject by intravenous administration. In some embodiments, the IL-2 therapy is administered to the subject by subcutaneous administration. In some embodiments, the IL-2 therapy is administered to the subject by intramuscular administration.

### 2. Schedule and Dosing

The IL-2 therapy may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 therapy is administered to a subject in need thereof about once every 8 hours, e.g., over a 5-day period or for about 14 doses. This period may be followed by about 9 days without administration of the IL-2 therapy. The cycle of administration about once every 8 hours, e.g., over a 5-day period or for about 14 infusions may then be repeated. After the second set of doses, there may be a rest period of about 7 weeks or more, followed by another treatment cycle or pair of treatment cycles.

In some instances, the IL-2 therapy is administered at a dose of 600,000 International Units/kg IV (e.g., per 8 hours during an administration schedule as described above).

In some embodiments, about 18 million International Units/m²/day IV are administered, e.g., by continuous IV infusion for two 5-day cycles. The cycles can be separated by a rest period of about 3-7 days. After the second cycle, there may be a rest period of about 7 weeks or more, followed by another treatment cycle or pair of treatment cycles.

In some embodiments, from about 1800 International Units/m2 up to about 18 million International Units/m² are administered once daily subcutaneously for 5 days, followed by a 2-day rest period. This schedule may be repeated weekly for about 6-8 weeks. Then, after about a 3-4 week rest period, the therapy may be resumed.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Immune Markers in Responders to High-Dose IL-2 (HD-IL-2)

### Patient Samples

Patients were retrospectively identified as having metastatic renal cell carcinoma (mRCC) and received treatment with HD-IL-2 (aldesleukin) therapy within the Levine Cancer Institute/Atrium Health hospital system (Charlotte, NC) between December 2009 and August 2013. Eligible patients had an available archived pre-treatment formalin-fixed paraffin embedded (FFPE) tumor tissue sample from a primary or metastatic site with sufficient material from which to extract RNA and DNA. A total of 36 patients met the study criteria.

Table 1 below shows the demographics of the study population, and Table 2 shows their disease outcomes and tumor characteristics.

In Table 1, the TNM Classification of Malignant Tumors was used to describe the size and location of the tumors, using “T” plus a letter or number (0 to 4). T1 means that the tumor was found only in the kidney and was 7 cm or smaller at its largest area. T2 means that the tumor was found only in the kidney and was larger than 7 cm at its largest area. T3 means that the tumor had grown into major veins within the kidney or perinephric tissue; however, it had not grown into the adrenal gland on the same side of the body as the tumor, and had not spread beyond Gerota's fascia. For the primary tumor, NA means that the tumor classification was not available. The “N” in the TNM Classification stands for lymph nodes. NX means that the regional lymph nodes could not be evaluated. NO means that the cancer had not spread to the regional lymph nodes. N1 means that the cancer had spread to regional lymph nodes. N2 means that the cancer had spread to distant lymph nodes (i.e., a lymph node in at least one part of the body other than near the kidneys). For the regional lymph nodes, NA means that the lymph node classification was not available.

TABLE 1
Demographics of Study Population.

| | Pre-Treatment | |
|---|---|---|
| Demographic | Biopsy (n = 36) | |
| Age, years, median (range) | 64 | (45-83) |
| Male sex, n (%) | 25 | (69%) |
| Race, n (%) | | |
| White | 33 | (92%) |
| Black | 3 | (8%) |
| Renal Cancer Type, n (%) | | |
| Clear cell | 30 | (83%) |
| Papillary | 3 | (8.5%) |
| Other | 3 | (8.5%) |
| Primary tumor (T), n (%) | | |
| T1 | 3 | (10%) |
| T2 | 5 | (16%) |
| T3 | 23 | (74%) |
| NA | 5 | (14%) |
| Regional lymph nodes (N), n (%) | | |
| N0 | 9 | (35%) |
| N1 | 4 | (29%) |
| N2 | 1 | (3%) |
| NX | 14 | (39%) |
| NA | 8 | (22%) |
| Eastern Cooperative Oncology Group (ECOG) | | |
| Performance Status, n (%) | | |
| 0 | 33 | (92%) |
| 1 | 3 | (8%) |
| Source of tumor for analysis, n (%)* | | |
| Primary tumor | 23 | (64%) |
| Biopsy of metastasis | 7 | (19%) |
| Mix of primary and metastatic tumor sample | 6 | (17%) |
| Time interval between tumor biopsy and IL-2 | 95 | (18-1803) |
| treatment start, days, median (range) | | |
| IL-2 therapy line of treatment, n (%) | | |
| 1^{st} | 32 | (89%) |
| 2^{nd} | 2 | (6%) |
| 3^{rd} | 2 | (6%) |
| Number of course of IL-2, n (%) | | |
| 1 | 29 | (81%) |
| 2 | 3 | (8%) |
| 3 | 3 | (8%) |
| 4 | 1 | (3%) |
| IL-2 Treatment Duration, days, median (range) | 24 | (2-199) |

*Adrenal gland biopsy was considered as a metastasis;

TABLE 2
Outcomes and Tumor Characteristics.

| | Pre-Treatment Biopsy | |
|---|---|---|
| Outcomes | (n = 36)* | |
| Median duration of follow-up, mos (95% CI) | 62.4 | (52.9-NR) |
| Best response, n (%) | | |
| Complete response (CR) | 1 | (3%) |
| Partial response (PR) | 12 | (34%) |
| Stable disease (SD) | 6 | (17%) |
| Progressive disease (PD) | 16 | (46%) |
| NA | 1 | |
| Objective response rate (ORR) | 37% | |
| Clinical benefit*, n (%) | 15 | (43%) |
| Yes | | |
| Median progression free survival (PFS)#, mos | 2.2 | (1.9-17.8) |
| (95% CI) | | |
| PFS at 6 months, (%) | 43% | |
| PFS at 1 year, (%) | 34% | |
| Median overall survival (OS)\hat{ }, mos (95% CI) | 67.6 | (31.6-NR) |

| | |
|---|---|
| OS at 6 months, n (%) | 97% |
| OS at 1 year, n (%) | 88% |

*Clinical benefit was defined as CR + PR + SD for >= 6 months.
#PFS was defined as time from IL-2 treatment initiation to progression or death and was also the duration of response.
\hat{ }OS was defined as time from IL-2 treatment initiation to death.
NR = not reached

### Clinical Annotation

Demographic and clinical variables were collected from medical records and entered into a dedicated auditable database (REDCap; www.project-redcap.org) designed around a pre-defined data dictionary. Data entry and subsequent quality control (QC) were performed by separate individuals. As appropriate, clinical variables were recorded at the time of initiation of IL-2 therapy. HD-IL-2 was given as a bolus IV injection (720,000 IU/kg) every 8 hours up to 14 doses followed by a 9-14 day rest period and then another week of systemic HD-IL-2 (up to 14 doses). Overall survival (OS) was defined as the interval from IL-2 treatment initiation to patient death. The Social Security Death Index was consulted whenever possible if death date was not available. Progression free survival (PFS) from IL-2 treatment was defined as the interval between initiation of initial IL-2 treatment and disease progression, or the date of death in the absence of noted disease progression. In cases where a patient was still alive or the date of death was unknown, date of last contact was used in place to estimate the censored OS/PFS. Clinical benefit was defined as complete response (CR), partial response (PR), or stable disease (SD). A total of 35 of 36 patients were evaluable for clinical response.

### RNA Sequencing

Hematoxylin and eosin (H&E)-stained FFPE sections underwent microscopic QC review by an anatomical pathologist to confirm histology diagnosis, evaluate percent tumor nuclei (≥5% required), percent necrosis, and cellularity prior to microdissection, and underwent dual DNA/RNA extraction using the truXTRAC FFPE total nucleic acid kit (Covaris). RNA quantitation was performed by Qubit measurement using ribogreen staining. RNA was qualitatively assessed for integrity by Agilent TapeStation gel electrophoresis. RNA samples approved for analysis (required 200 ng by ribogreen quantitation and a TapeStation DV200 value ≥20%) underwent library preparation using the Agilent SureSelect XT RNA direct prep kit. A no template control (NTC) and positive control sample (NA12878 FFPE RNA) were included in each run. Libraries were individually captured, reviewed for appropriate size using a Bioanalyzer or TapeStation trace, and quantified (KAPA library quantitation) prior to equal molar pooling. Sequencing was performed on an Illumina NovaSeq6000 sequencer using an S1 flow cell to generate about 50M, 2×100 bp paired end reads. RNA-Seq data were qualified and analyzed against other datasets within GeneCentric's archive. RNA sequencing was successfully performed on 35 of 36 patients.

### RNA Expression Analyses

Expression values for the samples were derived from raw RNAseq fastq files. Reads were aligned with STAR-aligner (GrCH38 ver. 22) to human assembly using the STAR/Salmon pipeline. Expression was quantified using the RSEM package and the GrCH38 human transcriptome reference. Genes were filtered for a minimum expression count (at least 10 reads in at least 5 samples), and for a protein coding annotation by Ensemble (final set of genes=16,901). Differential expression was assessed using the DESeq2 package on this filtered set of genes. For all other analyses, all expression values were log 2(1+x) transformed, median centered, and upper quartile scaled.

### Tumor Microenvironment Immune Activation

Investigation of immune differences by objective response category used previously published (Charoentong et al., Cell Rep. 18(1):248-262 (2017); Bindea et al., Immunity 39(4):782-795 (2013)) and proprietary (Faruki et al., J. Thorac. Oncol. 12(6):943-953 (2017)) immune markers that were calculated as average expression value (arithmetic mean) of all genes in each set of genes. Markers included: Activated B cell, Activated CD4 T cell, Activated CD8 T cell, Activated dendritic cell, CD56bright natural killer cell, CD56dim natural killer cell, Central memory CD4 T cell, Central memory CD8 T cell, Effector memory CD4 T cell, Effector memory CD8 T cell, Eosinophil, Gamma delta T cell, Immature B cell, Immature dendritic cell, Macrophage, Mast cell, MDSC, Memory B cell, Monocyte, Natural killer cell, Natural killer T cell, Neutrophil, Plasmacytoid dendritic cell, Regulatory T cell, T follicular helper cell, Type 1 T helper cell, Type 17 T helper cell, Type 2 T helper cell, IFNγ, and expression signatures based on 40 individual genes: Cd274(PD-L1), Pdcd1(PD-1), Pdcd1lg2(PD-L2), Ctla4, IL-2ra, IL-2rb, IL-2rg, Cxcl9, Cxcl10, Ifi27, Ifit1, Ifit2, Ifit3, Mx1, Mx2, Oas2, Stat1, Stat2, Stat3, Stat4, Stat5a(STAT5), Tbx21, Itga1(CD49A), Itgae(CD103), Cd28, Tnfrsf9(4-1BB), Cd40, Nfat5(NFAT), Type I Interferons (Ifna14, Ifna13, Ifna6, Ifna7, Ifna5, Ifna4, Ifna1, Ifna2, Ifna16, Ifnab1, Ifnk, Il6).

The sets of genes and individual genes were presented as heatmaps based on values generated using log 2 median-centered expression values of genes making up different immune markers and individual genes. Boxplots showing individual immune activation markers or individual gene expression levels were also created, and pairwise comparisons were conducted with p-values displayed when Wilcoxon Rank Sum Test p-values were <0.05. Heatmaps and box plots were generated using R program version 3.5.3. Box plots showed lower quartile, median and upper quartile expression data. Plot whiskers showed the full distribution of the expression data.

### Statistics

OS and PFS analyses were conducted using Cox-Proportional Hazards (CPH) model with right-censored endpoints. Associations between response to treatment and genomic markers were investigated using the Kruskal-Wallis test and Fisher's exact test for quantitative and qualitative markers. Association between response to treatment and clinical characteristics were evaluated using Fisher's exact test. Multivariable logistic regression models were used to test whether molecular subtype would predict response to treatment when adjusting for various genomic markers. All statistical analyses were conducted using R 3.6 software (http://cran.R-project.org).

Associations between categorical and continuous variables were evaluated using a t-test or analysis of variance (ANOVA), depending on the number of categorical variable levels.

### Results

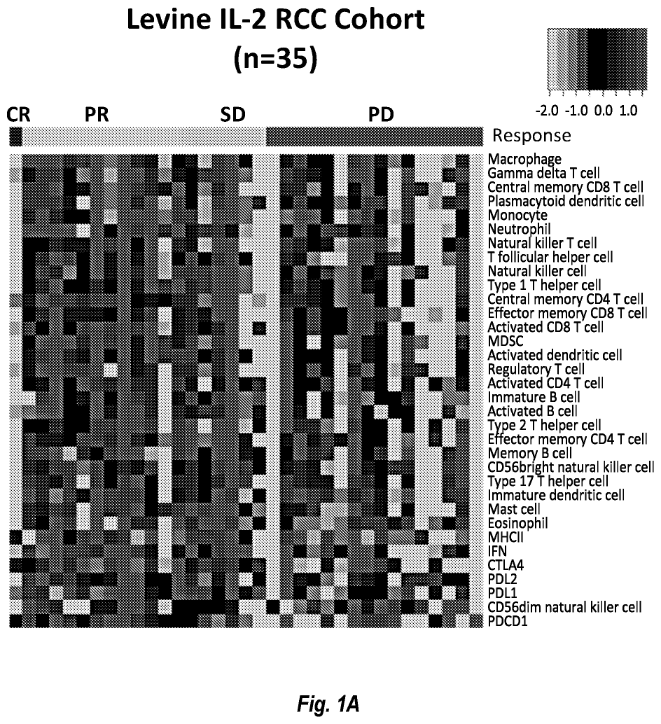

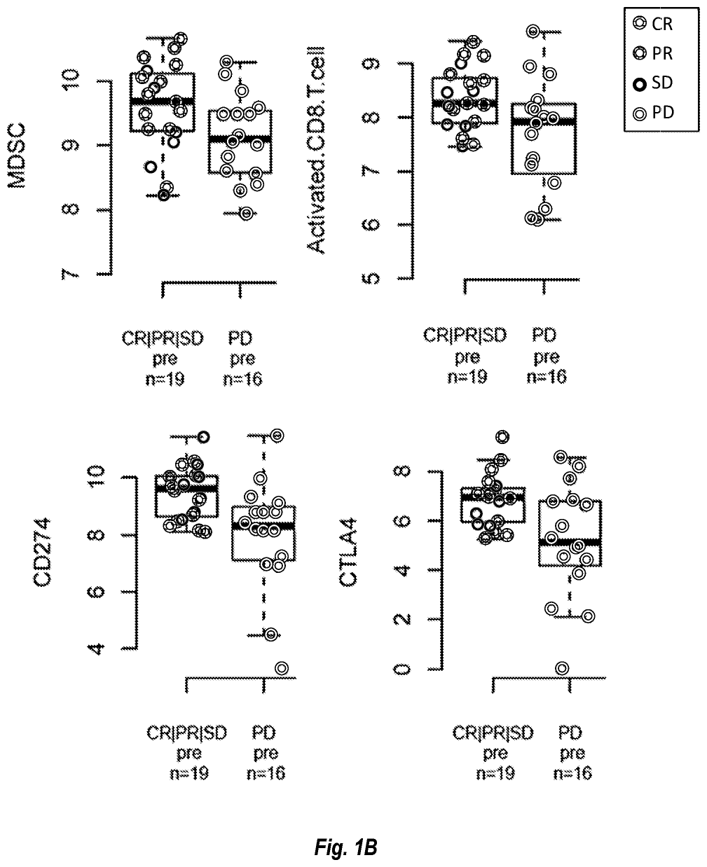

A select panel of immune markers, including sets of cell-associated genes and individual genes, were evaluated in the HD-IL-2 pre-treatment tumor specimens. Immune markers were increased in patients responding to the IL-2 therapy, compared to non-responders. FIG. 1A shows a heatmap showing expression of sets of cell-associated genes and individual genes (rows) in IL-2 pretreatment tumors (columns). The values are log 2 median-centered expression values. The sample annotation bar represents clinical response. FIG. 1B shows boxplots of sets of cell-associated genes and individual genes in pretreatment tumor samples with open circles colored by clinical response.

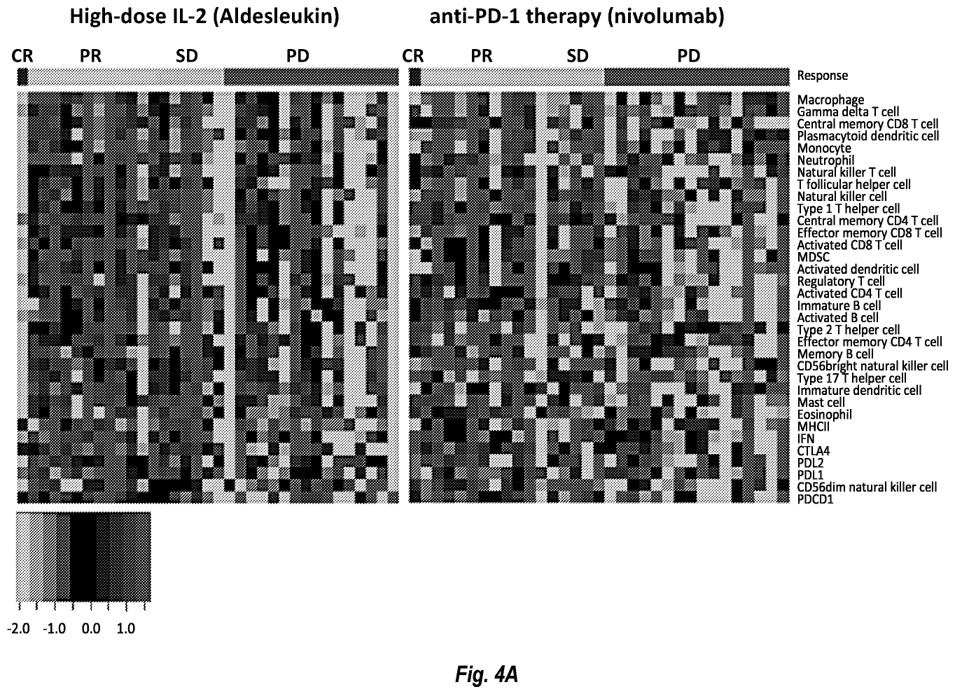

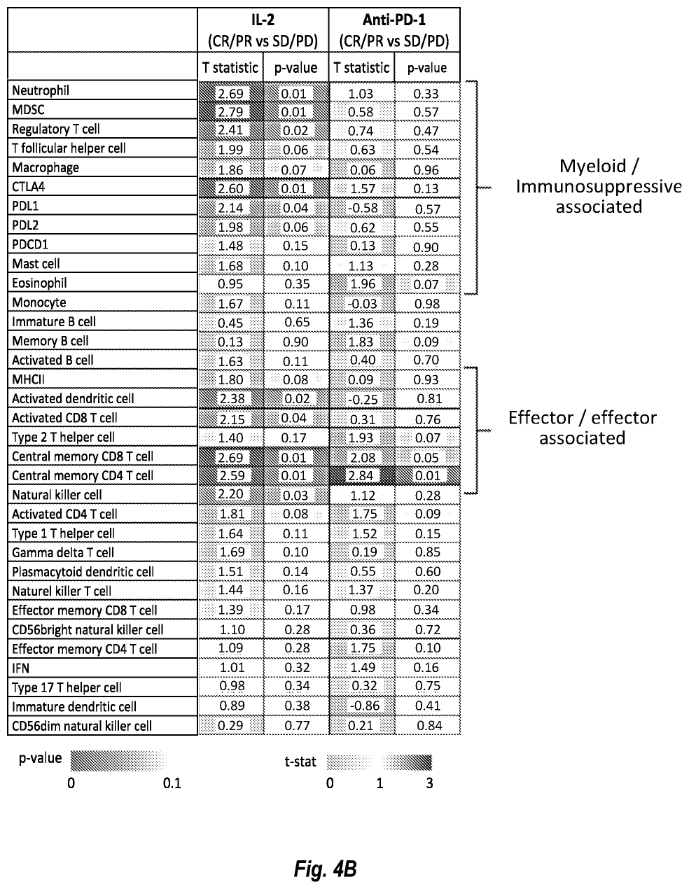

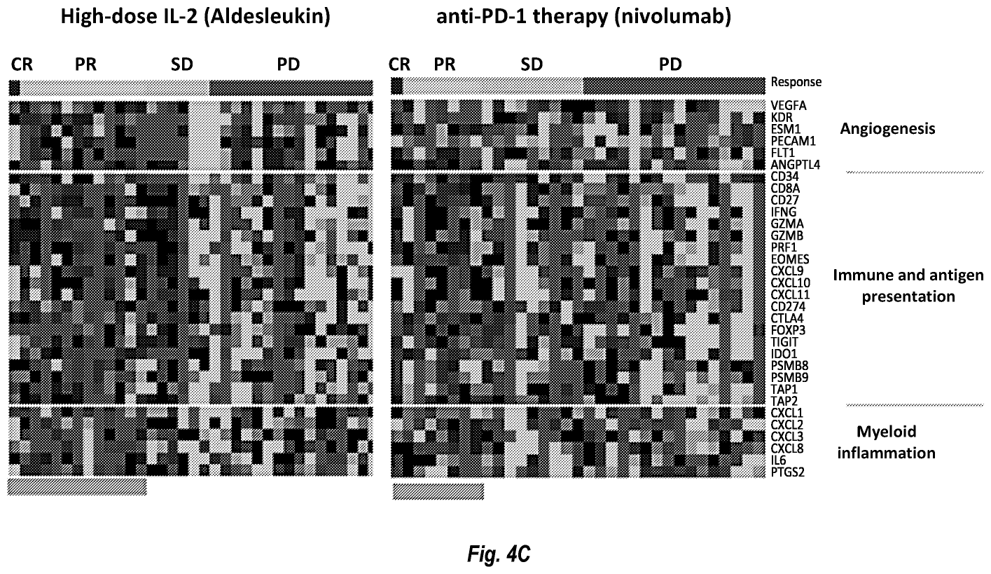

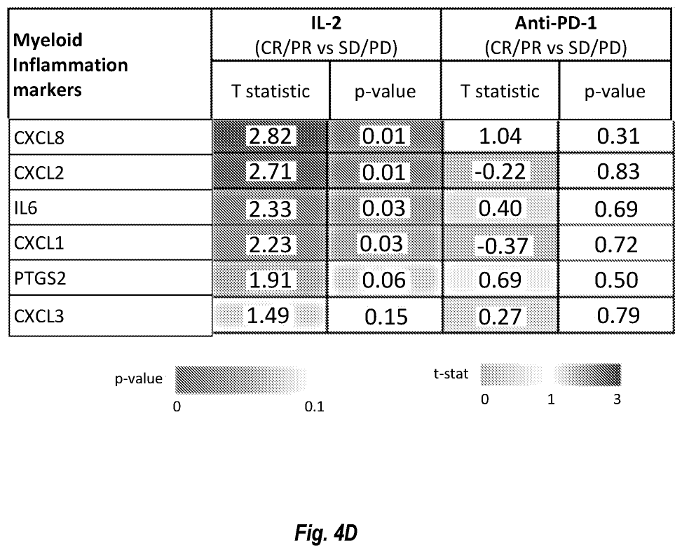

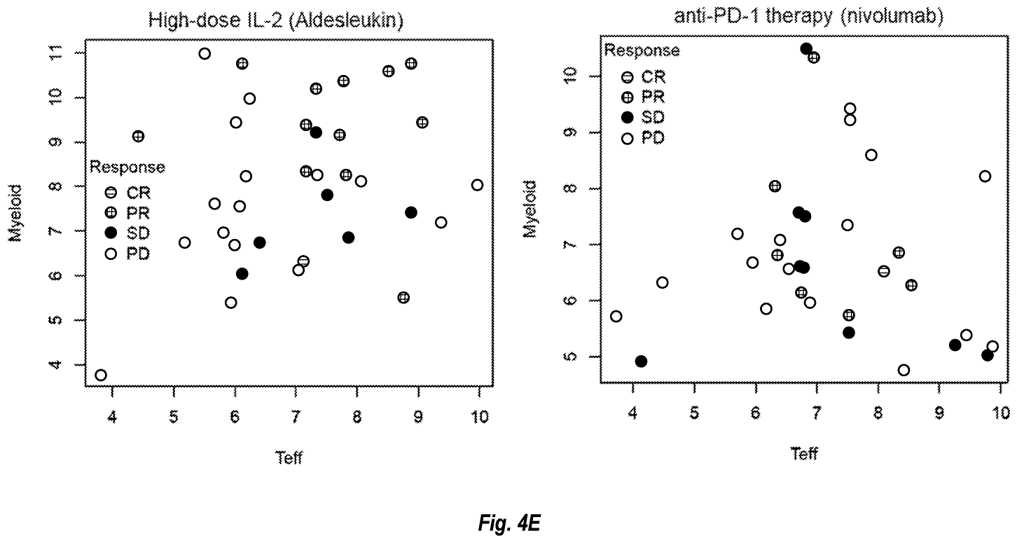

Most tumors associated with clinical benefit (CR, PR, SD) had significantly higher expression levels of most markers (FIG. 1 and FIG. 4B), suggesting high immune infiltration in responsive tumors. The elevated expression levels of immune markers in responsive tumors included several associated with immune suppression, such as sets of genes associated with myeloid-derived suppressor cells (MDSCs) (p=0.01), neutrophils (p=0.01), and regulatory T cells (p=0.02). Individual genes such as CD274 (PD-L1) (p=0.04) and CTLA4 (p=0.01), as well as sets of genes associated with effector cell types such as activated CD8 T cells (p=0.05) and natural killer (NK) cells (p=0.05), were also significantly elevated in responders based upon pre-treatment tumor samples.

### Example 2. Immune-Associated Gene Sets are Overrepresented in an Expression Classifier for HD-IL-2 Response

### IL-2 Treatment Response Nearest Centroid Classifier

To assess the immune markers findings in an orthogonal way and to initiate the process of prioritizing genes that could support the identification of patients poised to respond to IL-2 therapy, a nearest centroid classifier was developed for IL-2 therapy response (e.g., clinical benefit (CR/PR versus SD/PD)) using the 35 patients in Example 1 with clinical response data and corresponding tumor RNAseq expression profiles. Feature selection from the about 3,500 most highly expressed and high variance genes identified 40 markers that, based on cross-validation, were suitable for classifying the samples. The 40 markers included: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, KIAA1644, HLF, COL4A4, SLITRK4, SHISA9, IGSF9, TJP3, ZNF853, B4GALNT3, and ATP6V1B1.

### Results

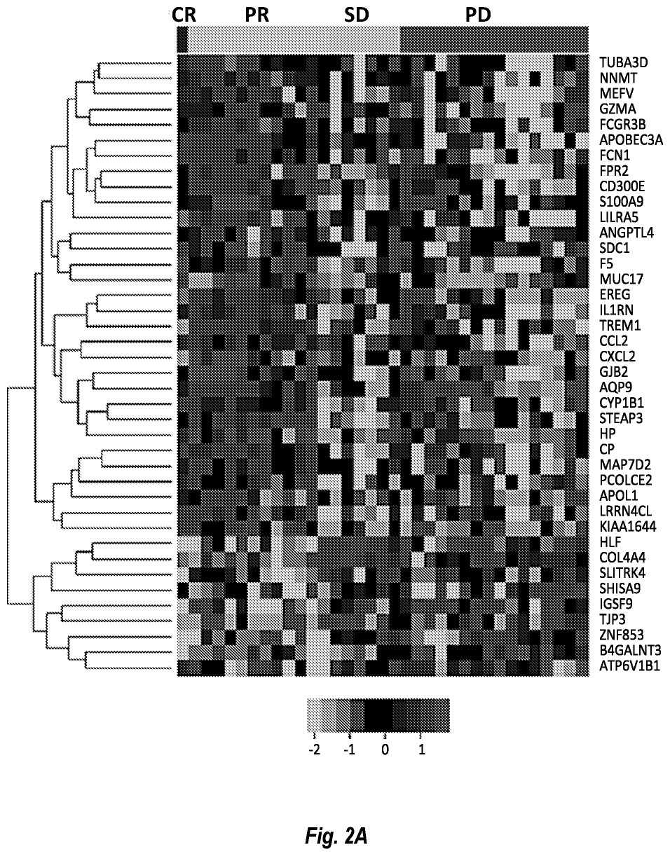

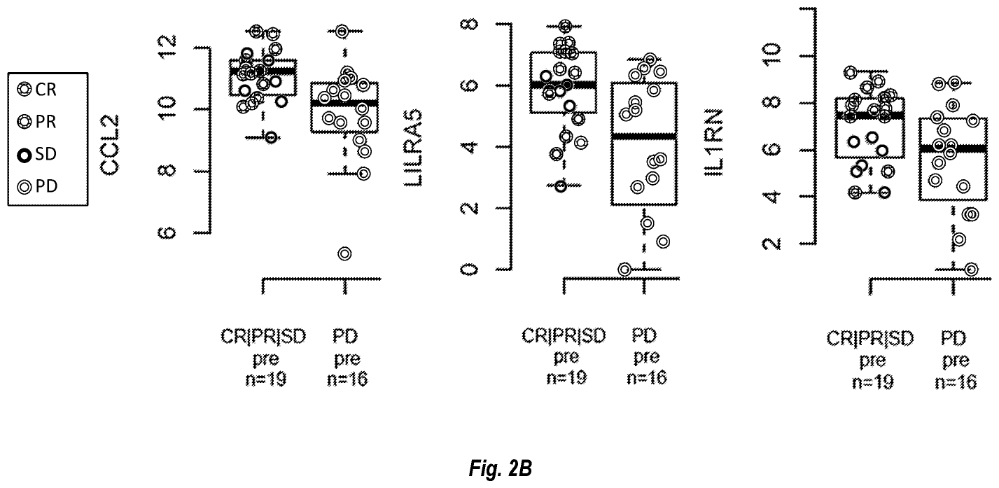

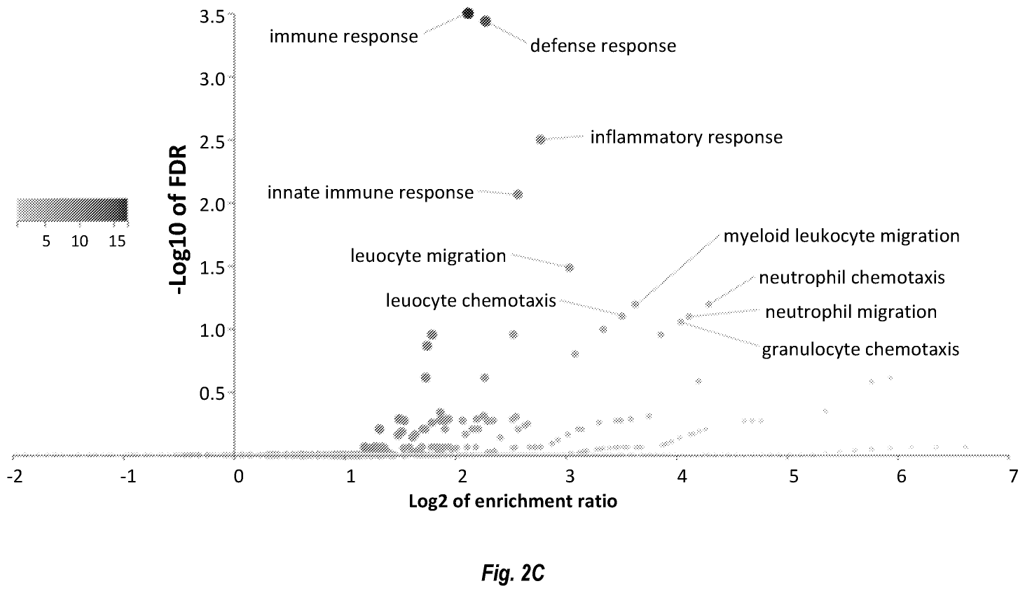

FIG. 2A shows a heatmap showing expression of 40 markers (rows) in the classifier in IL-2 pretreatment tumors (columns). Values are log 2 median-centered expression values. The sample annotation bar represents clinical response. FIG. 2B shows a boxplots of selected markers in pretreatment tumor samples with open circles colored by clinical response. FIG. 2C shows a volcano plot of overrepresentation analysis of the 40 markers in the classifier and the associated gene sets.

Although the number of patients (n=35 with evaluable clinical response data) limited the ability to divide the samples into training and test sets, the genes driving the classifier were reflective of the observed immune markers described above (FIG. 1). In the heat map of FIG. 2A, many markers of the classifier that are immune-associated were upregulated in samples associated with clinical response, such as CXCL2, CD300E, LILRA5, CCL2, and GZMA (selected box plots, FIG. 2B). Over-representation analysis of the 40 markers in the classifier exclusively resulted in immune-associated gene sets (FIG. 2C). The immune markers and the IL-2 response classifier suggest that patients demonstrating a clinical response to HD-IL-2 had higher pre-treatment levels of many immune-associated genes.

This classifier was subsequently evaluated in a larger Cancer Genome Atlas (TCGA) cohort of RCC patients undergoing a broad range of treatment modalities, revealing significant differences in survival between classifier positive and negative patients. Classifier positive and negative patients were identified based on nearest centroid analysis in which the first and second reference centroids were determined from the IL-2 non-refractory and IL-2 refractory subpopulations of the set of 35 patients analyzed herein, using the 40 markers shown in FIG. 2C.

### Example 3. Worse Overall Survival in RCC Based on Higher Immune Classification Methods in General but not in HD-IL-2 Treated Patients

RCC was historically notable for high immune-gene signatures failing to predict clinically favorable outcomes. This included intrinsic molecular subtypes based on gene expression, where the RCC subtype characterized by high lymphocytes and macrophages (clear cell B subtype or “ccB”) associated with worse overall survival. Further, the immune infiltration associated with poor prognosis was characteristically associated with immunosuppressive cell types such as those reflected by gene signatures (e.g., neutrophils, macrophages, and B-cells).

A 34-gene ccA/ccB renal cell carcinoma (RCC) classifier was developed based on Brooks et al., Eur Urol. 66(1):77-84 (2014), using calls on Cancer Genome Atlas Kidney Renal Clear Cell Carcinoma (TCGA KIRC) data (n=380) and a 34-gene list. Training was performed on ⅔ of the samples and testing on the remaining ⅓. Centroids were directly fitted using the centered training set and all available genes.

### Results

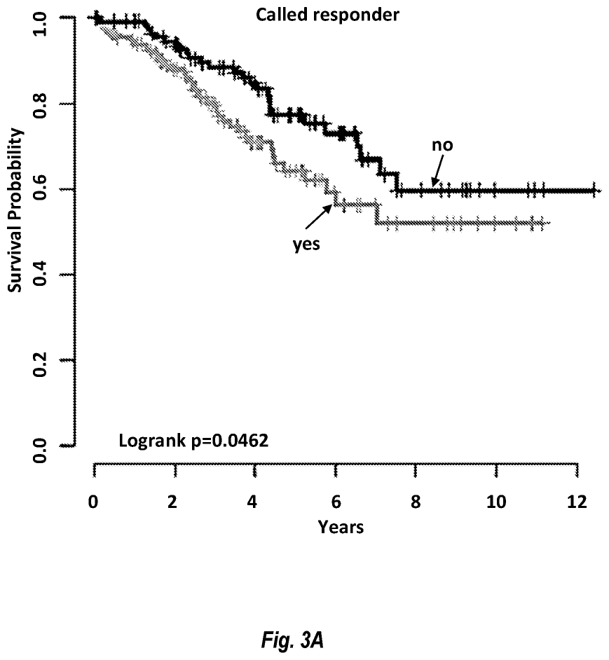

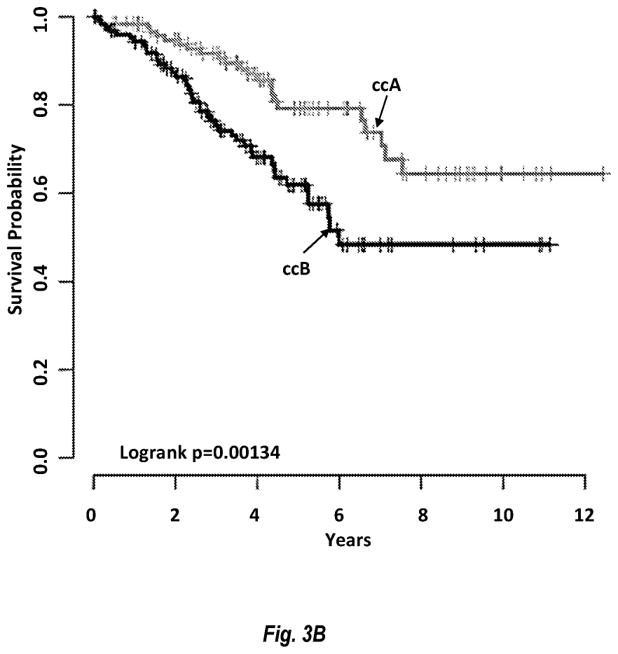

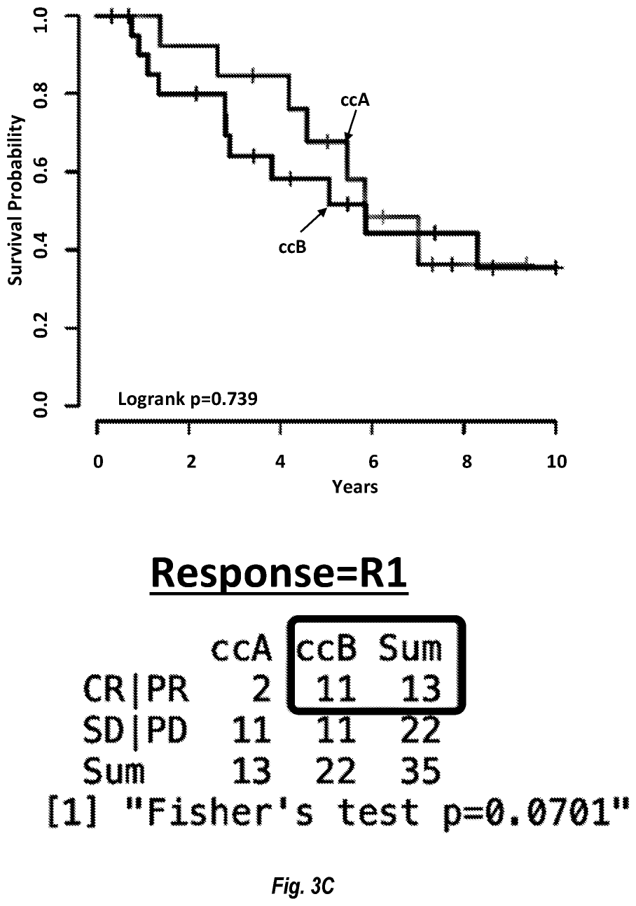

FIG. 3A shows a Kaplan-Meier plot of TCGA KIRC tumor samples (n=253) based on the 40-marker IL-2 response classifier of Example 2. “Yes” indicates tumor samples that were classified with the patients in the IL-2 cohort that have high immune infiltration and better clinical response. FIG. 3B shows a Kaplan-Meier plot of TCGA KIRC tumor samples (n=253) based on a canonical intrinsic subtype of RCC (the immune-high ccB). FIG. 3C shows a Kaplan-Meier plot of tumor samples from RCC patients treated with HD-IL-2 (n=36) based on a canonical intrinsic subtype of RCC (the immune-high ccB) along with a clinical response comparison.

Consistent with the association of the content of the IL-2 response classifier (trained on 253 randomized samples from TCGA KIRC samples), when applying the 34-gene ccA/ccB classifier to RCC patients from the TCGA, patients called responder “yes” had worse overall survival (p=0.05, FIG. 3A), which was similar to the worse overall survival in the immune-associated subtype ccB patients (p=0.001, FIG. 3B). However, and surprisingly, when the ccA/ccB classifier was applied to the HD-IL-2 treated RCC patients from the current study, the ccB subtype patients no longer had a worse overall survival (p=0.7, FIG. 3C), and a majority (11 of 13) of the patients with a clinical response (CR/PR) were within the ccB subtype (p=0.07). The closing of the survival curves for the HD-IL-2 patients and the trend towards a significant number of clinical responses (PR/CR) in the ccB subtype could be a result of “IL-2 rescue” in this subtype.

Thus, by several lines of inquiry, the markers identified in this RCC HD-IL-2 treated cohort projected to poor prognosis, consistent with the wide body of RCC literature, but also raised the possibility that an IL-2 therapy, such as HD-IL-2, could mitigate a poorly prognostic trajectory for select groups of RCC patients.

### Example 4. Immune Checkpoint Responders Displayed a Distinct Immunogenomic Profile from IL-2 Responders

Higher pre-treatment immune infiltration or immune markers such as PD-L1 were reported to be associated with response for several tumor types. However, for RCC, reports suggest a more nuanced pre-treatment immunogenomic profile of response where certain combinations of signatures or cell types are either predictive of non-response or response.

To assess the possibility of immunogenomic distinctions between anti-PD-(L)1 responders and HD IL-2 responders, RNA sequencing data from an anti-PD-1-treated cohort (with a similar distribution of response to the HD-IL-2 cohort) were compared to the HD IL-2 responders. Specifically, a separate dataset of whole transcriptome gene expression and clinical response data from a separate cohort of RCC patients treated with the anti-PD-1 inhibitor nivolumab was accessed as supplemental data from Miao et al., Science 359(6377):801-806 (2018), and comparted to the current HD-IL-2 cohort.

### Results

FIG. 4A shows heatmaps showing expression of sets of cell-associated genes and individual genes (rows) in IL-2 pretreatment tumors (columns in left heat map) and anti-PD-1 (right heat map). Values are log 2 median-centered expression values. The sample annotation bar represents clinical response. FIG. 4B shows statistical results for the sets of cell-associated genes and individual genes, comparing pretreatment responsive tumors (CR+PR) to non-responsive tumors (SD+PD) for IL-2 and anti-PD-1-treated patients. FIG. 4C shows heatmaps showing expression of markers (rows) in IL-2 pretreatment (columns, left heat map) and anti-PD-1 (right) tumors. Values are log 2 median-centered expression values. The sample annotation bar represents clinical response. The bright blue bars highlight the abundances of the myeloid inflammation genes for the responding tumors. FIG. 4D shows statistical results for the markers comparing pretreatment responsive tumors (CR+PR) to non-responsive tumors (SD+PD) for IL-2 and anti-PD-1-treated patients. FIG. 4E shows a scatter plot showing the relationship between the myeloid inflammation signature (Myeloid) and the T cell effector (Teff) signature for IL-2 and anti-PD-1 pre-treatment tumors, respectively.

In FIGS. 4A-4B, although the abundances across the response categories in the cohorts look similar, statistical analysis showed that the significance and t-statistic values are distinct. Both anti-PD-1 and IL-2 responders shared some effector cell signatures such as specific CD4 and helper T cell signatures (i.e., central memory CD4 T cell and Type 1 T helper cell signatures, respectively). In contrast, notable distinctions were observed, including signatures associated with immunosuppressive cell types that were among the most significantly elevated in the HD-IL-2 responders. Additionally, increased CD8 T cell expression (the set of activated CD8 T cell-associated genes) was increased in mRCC patients who responded to HD-IL-2 compared to non-responders, however this was not observed in patients responsive to anti-PD-1 treatment (FIG. 4B).

To assess anti-PD-(L)1 myeloid signature results in the context of the immunosuppressive myeloid markers observed in the HD-IL-2 cohort, the markers used in the IMmotion150 phase II trial (McDermott et al., Nat. Med. 24(6):749-757 (2018)) were assessed in the pre-treatment RCC specimens of both the HD-IL-2 and the anti-PD1 cohort. FIGS. 4C-4E reflects a similar profile to what was reported in the IMmotion150 trial, where the anti-PD-1-treated cohort responders had lower levels of the markers that define the immunosuppressive signature and appeared lower in the anti-PD1 cohort. The HD-IL-2 partial responders had higher levels of induction of these markers (FIG. 4C). The myeloid signature is comprised of 6 markers, with one of the markers, the myeloid chemokine CXCL2, being among the 40 genes in the response classifier of Example 2. The six markers in the myeloid classifier are mostly chemokines that play a central role in myeloid recruitment. Likewise, markers in the HD-IL-2 response classifier, such as CCL2, FPR2, L1RN, and S100A9, drive the gene set enrichments for factors such as myeloid leukocyte migration, neutrophil chemotaxis, and leukocyte chemotaxis. Thus, in addition to the shared CXCL2, these other shared myeloid features were differentially present in anti-PD-(L)1 and IL-2 responders.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

What is claimed is:

1. A method for treating a cancer in a subject, the method comprising:
administering an IL-2 therapy to the subject,
wherein in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644;
and the set of downregulated classification markers comprises at least one of HLF, COL4A4, SLITRK4, SHISA9, IGSF9, TJP3, ZNF853, B4GALNT3, and ATP6V1B1.

2. A method for treating a cancer in a subject, the method comprising:
selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644;
and the set of downregulated classification markers comprises at least one of HLF, COL4A4, SLITRK4, SHISA9, IGSF9, TJP3, ZNF853, B4GALNT3, and ATP6V1B1; and
administering an IL-2 therapy to the subject.

3. A method for selecting a subject with a cancer for an IL-2 therapy, the method comprising:
selecting the subject at least in part on the basis that in a sample from the subject a set of upregulated classification markers has elevated expression and/or a set of downregulated classification markers has decreased expression, wherein the set of upregulated classification markers comprises at least one of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644;
and the set of downregulated classification markers comprises at least one of HLF, COL4A4, SLITRK4, SHISA9, IGSF9, TJP3, ZNF853, B4GALNT3, and ATP6V1B1.

4. The method for the immediately preceding claim, further comprising:
obtaining the sample from the subject.

5. The method of any one of claims 3-4, further comprising:
extracting RNA from the sample.

6. The method of any one of claims 3-5, further comprising:
measuring expression of the set of upregulated classification markers and/or the set of downregulated classification markers in the sample.

7. The method of the immediately preceding claim, wherein the measuring of expression comprises sequencing RNA from the sample, conducting RT-qPCR with RNA from the sample, conducting a microarray analysis of the sample, or conducting a proteomic analysis of the sample.

8. The method of any one of the preceding claims, wherein the set of upregulated classification markers has elevated expression.

9. The method of the immediately preceding claim, wherein the set of upregulated classification markers comprises one of, two of, at least two of, three of, at least three of, four of, at least four of, five of, at least five of, six of, at least six of, seven of, at least seven of, eight of, at least eight of, nine of, at least nine of, ten of, at least ten of, 11 of, at least 11 of, 12 of, at least 12 of, 13 of, at least 13 of, 14 of, at least 14 of, 15 of, at least 15 of, 16 of, at least 16 of, 17 of, at least 17 of, 18 of, at least 18 of, 19 of, at least 19 of, 20 of, at least 20 of, 21 of, at least 21 of, 22 of, at least 22 of, 23 of, at least 23 of, 24 of, at least 24 of, 25 of, at least 25 of, 26 of, at least 26 of, 27 of, at least 27 of, 28 of, at least 28 of, 29 of, at least 29 of, 30 of, at least 30 of, or 31 of: TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644.

10. The method of any one of the preceding claims, wherein the set of downregulated classification markers has decreased expression.

11. The method of the immediately preceding claim, wherein the set of downregulated classification markers comprises one of, two of, at least two of, three of, at least three of, four of, at least four of, five of, at least five of, six of, at least six of, seven of, at least seven of, eight of, at least eight of, or nine of: HLF, COL4A4, SLITRK4, SHISA9, IGSF9, TJP3, ZNF853, B4GALNT3, and ATP6V1B1.

12. The method of any one of the preceding claims, wherein the set of upregulated classification markers comprises at least one of: GZMA, CD300E, LILRA5, IL1RN, CCL2, and CXCL2.

13. The method of the immediately preceding claim, wherein the set of upregulated classification markers comprises two of, at least two of, three of, at least three of, four of, at least four of, five of, at least five of, or six of: GZMA, CD300E, LILRA5, IL1RN, CCL2, and CXCL2.

14. The method of any one of the preceding claims, wherein the set of upregulated classification markers comprises at least one of: FPR2, S100A9, IL1RN, and CCL2.

15. The method of the immediately preceding claim, wherein the set of upregulated classification markers comprises two of, at least two of, three of, at least three of, or four of: FPR2, S100A9, IL1RN, and CCL2.

16. The method of any one of the preceding claims, wherein the set of upregulated classification markers comprises at least X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

17. The method of any one of the preceding claims, wherein the set of upregulated classification markers comprises at least 2X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

18. The method of any one of the preceding claims, wherein the set of upregulated classification markers comprises at least 3X of TUBA3D, NNMT, MEFV, GZMA, FCGR3B, APOBEC3A, FCN1, FPR2, CD300E, S100A9, LILRA5, ANGPTL4, SDC1, F5, MUC17, EREG, IL1RN, TREM1, CCL2, CXCL2, GJB2, AQP9, CYP1B1, STEAP3, HP, CP, MAP7D2, PCOLCE2, APOL1, LRRN4CL, and KIAA1644 and at the set of downregulated classification markers comprises at least X of HLF, COL4A4, SLITRK4, SHISA9, IGSF9, TJP3, ZNF853, B4GALNT3, and ATP6V1B1, where X is 2, 3, 4, 5, 6, 7, 8, or 9.

19. The method of any one of the preceding claims, wherein the cancer is renal cancer.

20. The method of any one of the preceding claims, wherein the cancer is renal cell carcinoma.

21. The method of any one of the preceding claims, wherein the cancer is renal cell carcinoma of subtype clear cell B (ccB).

22. The method of any one of the preceding claims, wherein the cancer is metastatic.

23. The method of any one of the preceding claims, wherein the subject has not received any prior IL-2 therapy.

24. The method of any one of the preceding claims, wherein the IL-2 therapy comprises a polypeptide having at least 80%, 85%, 90%, 95%, 96%, 97%, 98%, 99%, or 100% sequence identity to SEQ ID NO: 1.

25. The method of any one of the preceding claims, wherein the IL-2 therapy comprises aldesleukin.

26. The method of any one of the preceding claims, the IL-2 therapy is administered to the subject by subcutaneous administration.

27. The method of any one of the preceding claims, the IL-2 therapy is administered to the subject by intravenous administration.

28. The method of any one of the preceding claims, further comprising refraining from administering a PD-1-targeting therapy or PD-L1-targeting therapy to the subject.

29. The method of any one of the preceding claims, further comprising refraining from administering a PD-1-targeting therapy to the subject.

30. The method of any one of the preceding claims, wherein the sample comprises a tumor tissue.

31. The method of any one of the preceding claims, wherein the sample comprises a primary tumor tissue or a metastatic tumor tissue.

32. The method of any one of the preceding claims, wherein the sample comprises a formalin-fixed paraffin embedded (FFPE) tumor tissue.

33. The method of any one of the preceding claims, wherein the sample comprises immune tissue.

34. The method of any one of the preceding claims, wherein the set of upregulated classification markers has elevated expression and/or the set of downregulated classification markers has decreased expression according to nearest centroid analysis.

35. The method of the immediately preceding claim, wherein the nearest centroid analysis comprises determining first and second distances; the first distance is between a sample vector comprising expression values of the set of upregulated classification markers and/or the set of downregulated classification markers and a first reference centroid of the set of upregulated classification markers and/or the set of downregulated classification markers determined from expression values from IL-2 non-refractory subjects; and the second distance is between the sample vector and a second reference centroid determined from expression values of the set of upregulated classification markers and/or the set of downregulated classification markers from IL-2 refractory subjects.

36. The method of any one of the preceding claims, wherein elevated expression and/or decreased expression are determined using logarithmic (e.g., log 2) median-centered expression values for the set of upregulated classification markers and/or the set of downregulated classification markers.

37. An IL-2 therapy for use in the method of any one of the preceding claims.

38. Use of an IL-2 therapy for the manufacture of a medicament for the method of any one of claims 1-36.
